SEQUENCE LISTING (2 sequences)

SEQ ID NO: 1 - DNA, 24 bp, Artificial Sequence
  Oligonucleotide
aaatacgagatccaccgagactcc

SEQ ID NO: 2 - DNA, 22 bp, Artificial Sequence
  Oligonucleotide
gcaggaacagcgccgatacaat

(19) United States
(12) Patent Application Publication
(10) Pub. No.: US 2010/0009348 A1
(43) Pub. Date: Jan. 14, 2010

(54) Methods and Constructs for Analyzing Biological Activities of Biological Specimens and Determining States of Organism

(75) Inventors: Sergei Romanov, Chapel Hill, NC (US); Sergei Makarov, Chapel Hill, NC (US)

Correspondence Address: JONES DAY, 222 EAST 41ST ST, NEW YORK, NY, 10017, US

(73) Assignee: ATTAGENE, INC.

(21) Appl. No.: 11/992,900

(22) PCT Filed: Sep. 27, 2006
(86) PCT No.: PCT/US2006/038175

§ 371 Date: May 4, 2009

Related U.S. Application Data
(60) Provisional application No. 60/721,860, filed on Sep. 28, 2005.

(51) Int. Cl. C12Q 1/68; C12Q 1/02; A01K 67/027; G01N 33/68

(52) U.S. Cl. 435/6; 435/29; 800/3; 435/40.5; 436/86

(57) ABSTRACT

This application provides methods of determining biological activities of a biological sample comprising, for example, comparing the profile of transcription factor activities in a cell contacted with the biological sample to a control profile, such as a profile of transcription factor activities in a cell not contacted with the biological sample.

### 1. RELATED APPLICATIONS

This application claims the benefit of U.S. Provisional Application No. 60/721,860, filed Sep. 28, 2005, which is incorporated herein by reference in its entirety for all purposes.

### 2. FIELD OF THE INVENTION

This application relates to methods of analyzing transcriptional activities of transcription factors and cis-regulatory elements in a cell, for example, to determine a biological activity in a sample applied to the cell.

### 3. BACKGROUND

In multicellular organisms, cells communicate by releasing myriads of signals, such as neuromediators, hormones, growth factors, cytokines, etc. These mediators carry specific instructions as to how particular cell types, organs, and tissues, should alter their behavior.

States of the host (e.g., health vs. disease) can be analyzed by assessing the spectra of biological activities in its biological fluids in regard to their actions on different cell types and tissues.

Several approaches for analyzing the content of biological fluids and other samples are being developed, such as proteomics that evaluates concentration profiles of proteins in biological specimens, e.g., by using antibody arrays, and metabolomics, wherein profiles of biological mediators are evaluated according to their weights and molecular structures, e.g., by using chromatography, mass-spectrometry, etc. However, analyzing the physical-chemical properties of individual constituents provides little information about the biological activities of evaluated samples.

Biological activity can be directly assessed by using various cell-based assays, where analyzed samples are contacted with tester cells in culture and phenotypical changes (e.g., apoptosis, proliferation, differentiation) of tester cells are evaluated. However, as there are many distinct molecules that can elicit same phenotypical changes in tester cells, those assays can be used for detecting certain (e.g., pro-apoptotic, mitogenic, etc.) activities but are poorly suited for analyzing the complex spectra of biological activities in the samples.

A cell-based assay recently proposed characterizes evaluated biological samples according to the alterations in gene expression occurring in tester cells in response to contact with the sample. In this approach, the response of the tester cells is analyzed by assessing the profile of gene expression (transcriptome) in these cells, e.g., by hybridizing cellular RNA to detection array (USPTO publication No. 2005/0181354 A1).

Such an approach, however approach has several shortcomings. One is that analyzing the transcription response requires the analysis of expression of tens of thousands genes. Another challenge is how to interpret the large amounts of data produced by microarrays. To find the characteristic patterns in expression of thousands of genes, algorithms have been developed that allow identifying the clusters of genes regulated in a similar fashion (see, e.g., Hughes et al., 2000, J. Mol. Biol. 296:1205-1214) but this problem still requires further integration of higher-order statistical analyses and data management. Thus, such assays are laborious, expensive, and their results are difficult to interpret in regard of biological activities of analyzed samples.

### 4. DESCRIPTION OF INVENTION

An alternative to describing cells at the level of gene expression (i.e., transcriptiomics) is to investigate the molecular changes occurring at a signal transduction level. In response to cell stimulation, cell activates the signal transduction pathways that result in alteration of gene expression. At the apex of most signal transduction pathways lay inducible transcription factors (TFs), the proteins that bind specific DNA sequences within the promoter regions of genes, thereby initiating or repressing transcription. Activity of TFs is regulated at many levels, such as post-translational modification (e.g., phosphorylation or acetylation), degradation, nuclear translocation, DNA binding, and/or by interactions with other proteins, including the basal transcriptional machinery, co-activators or co-repressors, and other TFs. These different levels of regulation allow gene expression to be tightly controlled. Through different combinations of these regulatory mechanisms, eukaryotes are able to elicit a myriad of gene expression patterns.

An approximate 2,000 different TFs that comprise several hundreds of distinct TF families exist in the human genome. These TF families orchestrate the expression of tens of thousands genes. As the complexity of cell regulation is dramatically reduced at the TFs level, analyzing the molecular changes at the TFs level enables much simpler and comprehensive interpretation of biological activities of tested compounds.

The present invention provides methods whereby the spectra of biological activities of biological specimens are analyzed by assessing their effects on the signal transduction network in tester cells. As readout of these activities, the present invention provided methods of analyzing the activities of transcription factors (TFs), as well as the transcriptional activities of cis-regulatory response elements (cisREs) that are regulated by these TFs. Advantages of this invention include, for example, that the functional states of organism are characterized by analyzing the biological activities of biological samples derived from the organism. This obviates the necessity of introducing reporter systems into evaluated host and thus provides the opportunity of a non-invasive assessment. Furthermore, the invention affords the assessment of collections of archived materials, e.g., serum, tissues, etc. The present invention provides methods of analyzing the transcriptional activities of TFs and cisREs, methods of deriving information about the biological activities of various biological specimens and methods of identifying selective markers of disease, evaluating drug candidates, discovering the targets for therapeutic treatments, among many other biomedical applications.

The biological activity of analyzed sample is defined through the ability of the sample to induce changes in activities of signal transduction pathways in tester cell system hereafter called biosensor. The alterations in the activities of the signals transduction pathways are evaluated by assessing the profiles of activities of TFs and/or cis-REs in these biosensors.

The invention is based, in part, on the premises that:
(i) the state of biological system can be characterized by analyzing the biological activities of its constituents (e.g., biological fluids, tissue extracts, or other specimens);
(ii) the biological activities in analyzed sample can be assessed by contacting the sample with a tester cell system (hereafter termed biosensor) and by determining alterations in signal transduction within the biosensor;
(iii) the alterations in signal transduction can be comprehensively described by assessing profiles of activities of multiple transcription factors (TFs) or profiles of activities of reporter constructs that contain cis-response elements (cisREs) controlled by those TFs;
(iv) sufficient resolution of biosensors can be achieved to distinguish different states of analyzed biological system.

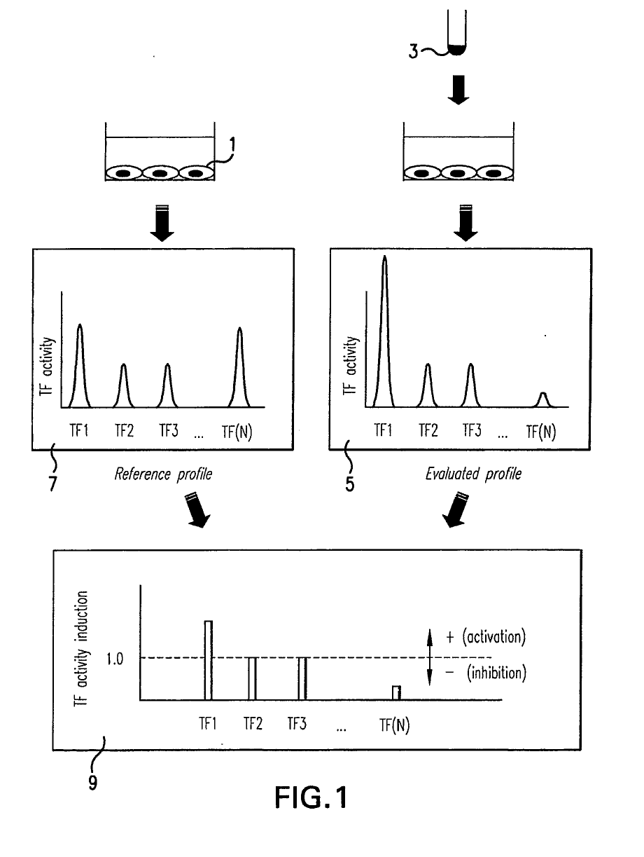

FIG. 1 depicts one ramification of the invention. In this example, biosensor 1 is a homogenous population of one cell type that is maintained in culture under standard growth conditions. The biosensor is contacted with analyzed sample 3 by adding the analyzed sample to the growth medium for a defined period of time. At the end of incubation, a determination of the profile of activities of TFs in the biosensor is made and thus determines the evaluated TF activity profile 5. A determination of the reference profile of activities of the TFs 7 in the biosensor that was not contacted with the evaluated sample can be made. By comparing the profiles of TF activities (evaluated vs. reference), one determines the changes in activities of individual TFs occurring in response to the analyzed sample. The resulting profile of alterations of TF activities 9 represents a molecular signature of the biological activity of the evaluated sample 3.

Activities of TFs within the biosensor can be determined by using different approaches.

In one embodiment, a TF activity is assessed by measuring the binding activity of the TF to a DNA probe comprising a TF-binding sequence. This can be done by assaying cellular extracts in any available DNA binding assay, e.g., a gel-shift assay (also known as electromobility shift assay, or EMSA), an ELISA-based DNA binding assay, etc.

In alternative embodiment, the transcriptional activity of TFs, i.e., the ability to activate the expression of target genes, are evaluated. To do so, biosensor cells are supplied with a library of reporter constructs enabling the assessment of multiple TFs and cisREs, and the activities of evaluated TFs are assessed by analyzing the activities of corresponding reporter constructs. Many reporter constructs are available for this purpose, e.g., luciferase, CAT, GFP reporters, etc. The activities of multiple TFs can be assessed in parallel by using libraries of reporter RNA constructs (U.S. patent publication No. 2006/0160108).

By comparing the signature of evaluated sample with a database comprising the molecular signatures of other samples, one can relate the evaluated sample to other samples. To this purpose, mathematical algorithms exist that can quantitatively compare the TF activity profiles, e.g., correlation analysis. Various parametric and non-parametric metrics are available for this purpose, e.g., Euclidian distance, Pearson's correlation coefficient, rank order correlation, etc.

The resolution can be defined as the capability to distinguish between different biological activities. For example, many distinct molecules exist, e.g., interleukin-1 (IL-1), tumor necrosis factor alpha (TNFa), bacterial lipopolysaccharide (LPS), that can induce the activation of the transcription factor NF-kB. Thus having observing the activation of NF-kB per se cannot distinguish between those mediators. However, in many circumstances, it is important to have a capability to distinguish those molecules. For example, the presence of TNFa and IL-1b in circulation may indicate chronic inflammatory disease or endotoxemia, while the presence of LPS is indicative of endotoxemia.

There are several distinct approaches whereby the resolution of the assay can be further optimized.

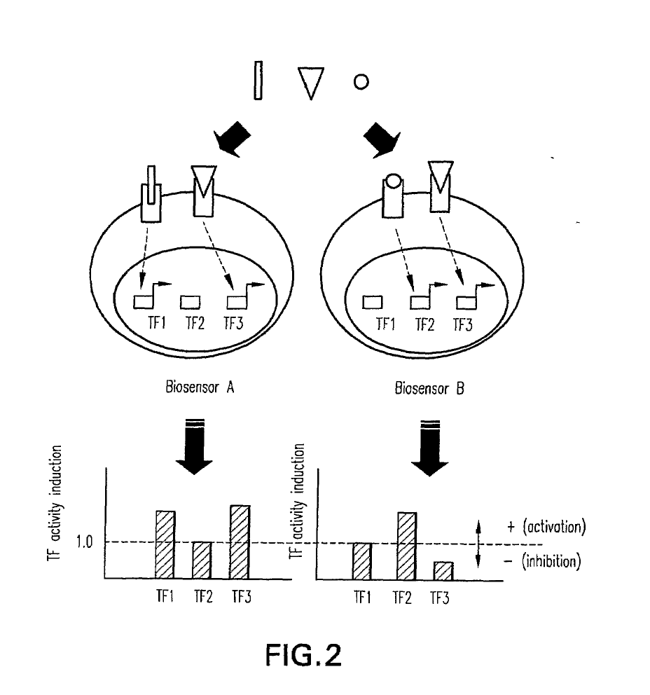

In one approach, the sample is analyzed by using a panel of biosensors that represent different cell types. That is, the molecular signatures of the sample are assessed by contacting the sample with biosensors representing epithelial cells, immune cells, fibroblasts, neural cells, etc., and the profiles of TF activities in those cells are assessed. It is anticipated that the molecular signatures will be different in different cell types. For example, LPS will activate NF-kB in the cells that express an LPS receptor (e.g., TLR-4), but not in the cells that lack this receptor. Similarly, the presence of TNFa in the sample will activate NF-kB in cells that express TNFa receptors, but not within cells lacking these receptors, etc. Therefore, in order to distinguish between two different biological activities, one should expand the panel of biosensors by including different cell types until the desirable resolution is achieved (FIG. 2).

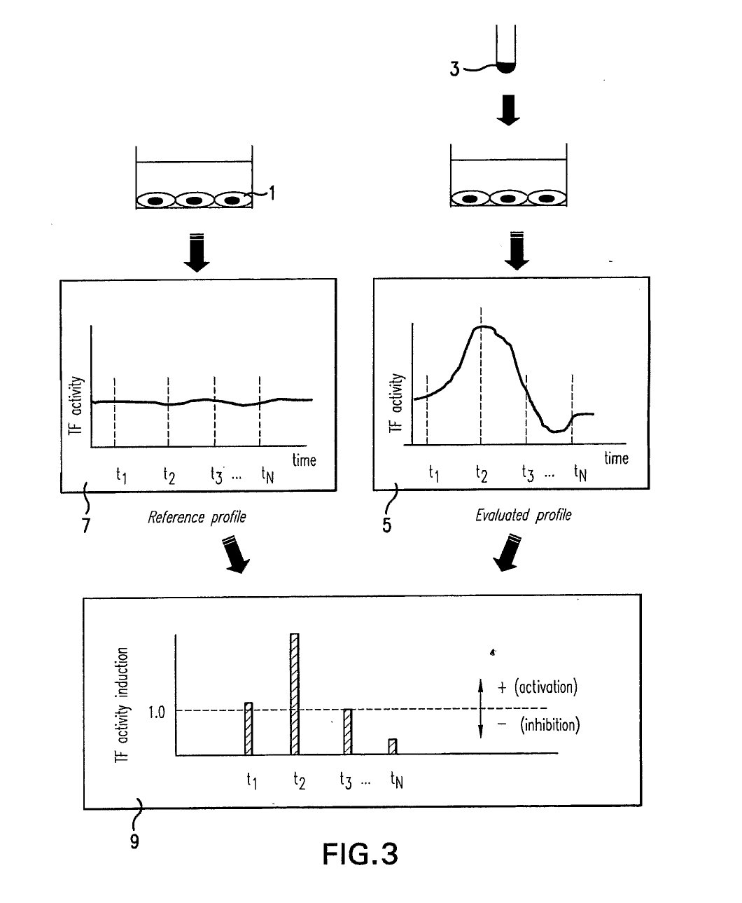

In another approach, differential biological activities can be distinguished by analyzing temporal patterns of TF activity profiles. For example, TNFa and IL-1b induce a very rapid activation of NF-kB that reaches a peak within minutes and then subsides. Two to three hours later, the second wave of NF-kB activation occurs. By contrast, platelet-derived growth factor (PDGF) causes a slow activation of NF-kB that reaches a peak within hours of stimulation (Romashkova and Makarov, 1999). Therefore, in order to distinguish between two different biological activities, one can compare the profiles of TF activities at various time points after the contact with the samples (FIG. 3).

In yet another approach, the resolution can be increased by increasing the number of TFs that are assessed in the assay. For example, LPS activates both NF-kB and interferon-response elements, while TNFa activates only NF-kB. Thus, by assessing the activities of NF-kB and IFNg-responsive elements, one can distinguish between LPS and TNFa in the analyzed sample. Therefore, in order to distinguish between two different biological activities, one should expand the number of evaluated TFs until desirable resolution is achieved.

In yet another approach, biosensor is contacted with analyzed sample in the presence of a response-modifying agent. For example, inflammation often results in the release into circulation of anti-inflammatory molecules, such as IL-1 receptor antagonist, corticosteroids, soluble TNFa receptors, etc. These molecules may not necessarily induce alterations in signal transduction within biosensor, but may selectively prevent the activation of TFs by cytokines. For example, if evaluated sample inhibits activation of NF-kB in response to cytokine IL-1, but not in response to TNFa, this may indicate the presence of selective inhibitors of IL-1. In contrast, a selective suppression of TNFa-inducible NF-kB activation by the evaluated sample will indicate the presence of selective inhibitors of TNFa. Thus, by combining the evaluated sample with response-modifying agents, one can increase the resolution of assay. Various agents can be used as response-modifying agents, such as cytokines and mixture of cytokines, growth factors, low-molecular weight compounds, radiation, etc. Also, the TF activity profile in biosensors can be altered by using various expression constructs, e.g., by expressing cDNAs encoding various genes, as well as dominant-negative and constitutively active variants of those genes. Furthermore, many different ways that alter gene expression within biosensors can be used, including antisense molecules, small interfering RNAs, etc. Therefore, to distinguish between two different biological activities, one evaluates the biological activities of samples in the presence of various response-modifying agents, until desirable resolution is achieved.

Various biological systems can be used as biosensors. For example, biosensor can be a homogenous cell culture comprising one cell type. The biosensor can also comprise a mixed population of different cell types. Biosensor can also comprise a tissue or an organ culture, e.g., brain slice culture, liver slice culture, skin flap, etc. Also, a cell population, organ, or tissue can be engrafted into animals to serve as an in situ biosensor. For example, the engrafted tissue, organ, or cell population can be supplied with a library of reporter constructs, and the monitoring of reporter constructs' expression will provide information about biological activities of fluids and tissues contacting the engrafts. Whole organs and tissues of live animals can also be used as biosensors. For example, an isolated liver of live animal can be perfused with analyzed sample followed by assessment of TF activity profiles within the liver, or skin of animal can be contacted with analyzed sample, followed by assessment of TF activity profiles within the skin.

Various biological samples can be analyzed by this invention, e.g., biological fluids, including saliva, blood, serum, cerebrospinal fluid, synovial fluid, urine, semen, breast milk, bile, tears, feces extracts, etc., as well as extracts, concentrates, components, or fractionates thereof. One can also analyze biological activities of cellular and tissue extracts, conditioned cell culture medium, etc. Furthermore, various cells can also be considered as biological samples. In this regard, the biological activity can be defined as the alteration in signal transduction that occurs upon cell-cell contacts of biosensors with the samples, i.e., live or fixed (e.g., glutaraldehyde-fixed, formalin-fixed) cells, or cell membranes.

The present invention provides means to characterize functional state of a biological system thru assessment of biological activities of its constituents. Variety of biological systems can be characterized in this way, including cell cultures, mixed population of cells, tissue and organ cultures, engrafted cells and tissues, organs and tissues of live animals, or whole live animals. To characterize the biological system, one collects biological samples from this system (cell supernatants, tissue extracts, bodily fluids, etc.), contacts these samples with biosensors, and determines alterations in signal transduction in the biosensors (i.e., alterations in profiles of TFs activities).

The invention is further useful for the identification of markers of perturbed functional states of various biological systems. For example, perturbed state of an animal can be a disease. To determine markers of the disease, one assesses biological activities of one or several biological samples from the diseased animal and the biological activities in corresponding samples from undisturbed (healthy) animal, and, by comparing biological activities in these samples, one identifies markers of the disease. For example, if serum of animal with a certain disease induces activation of certain TFs in biosensors, while serum of healthy animals does not, then activation of these TF provides a marker of the disease. The differentially inhibited TFs also provide the markers of the disease.

Furthermore, one can assess the intensity of perturbation (e.g., the severity of a disease) by quantitatively evaluating the intensity of the marker of said perturbation.

Different kinds of perturbations can be assessed, including a disease, a pre-disease state, aging, different treatments that alter the functional state of biological system, e.g., stress, diet, therapeutic treatment, administration of chemical compounds, toxins, pathogens, etc.

The invention establishes method of identifying markers that distinguish different perturbed functional states of a biological system. To do so, one determines the biological activities of one or multiple biological samples derived from the biological system in one perturbed state and in another perturbed state of organism, and, by comparing those biological activities, identifies the markers that distinguish those perturbed states.

The invention further defines method of diagnostics of disease and pre-disease states of an organism. To do so, one determines the biological activities in one or multiple biological samples derived from the evaluated organism and compares these biological activities with database of markers of diseases and pre-disease states. Various diseases can be diagnosed in this way, including chronic inflammatory diseases (e.g., arthritis, lupus, etc.), metabolic diseases (such as diabetes), various cancers, neurodegenerative diseases (e.g., Alzheimer disease, Parkinson disease, etc.), psychosomatic disease, various infections (e.g., bacterial and viral infections), hereditary diseases (e.g., premature aging), pre-infarction state, pre-diabetic state, etc.

The invention further defines method of identification of putative therapeutic targets and drug candidates for various diseases and pre-diseased states. To do so, one identifies markers of said disease and pre-disease states. As discussed above ([0028]), those markers represent TFs that are upregulated or downregulated in biosensors by contact with samples derived from diseased organisms. Therefore, these markers represent putative therapeutic targets. For example, if a marker of the disease is upregulated NF-kB activity, then inhibitors of NF-kB,-or inhibitors of the upstream signal transduction cascades controlling NF-kBF,-represent a putative treatment for the disease. Vice versa, if a certain TF is inhibited, activators of this TF may represent a putative treatment.

Akin to that, present invention defines method of evaluating the efficacy of drug candidates and other treatments for a disease.

### 5. EXAMPLES

The working examples below demonstrate the successful assessment of biological activities of sera derived from healthy and diabetic animals, using different types of reporter cells to determine non-redundant set of biological activities present in the serum of diabetic animals, and how information can be assembled to identify a disease.

### 5.1. Example 1

Materials and general procedures used in the examples that follow are described below.

Animals. Manipulations with experimental rats were performed in certified animal facility and according to the approved animal protocol.

Cells. Human hepatocellular carcinoma, HepG2, embryonic kidney epithelial, HEK293 and rat insulinoma, U7, cell lines were maintained on DME media (Invitrogen, Carlsbad, Calif., USA) supplemented with 10% FBS (HyClone, Logan, Utah, USA) and further supplemented with antibiotics. Streptozotocin was purchased from Sigma (Sigma-Aldrich, St. Louis, Mo., USA).

Plasmid DNA manipulations. Manipulations with plasmid DNAs were performed using standard molecular biology techniques known in the art, as described, for example, in Sambrook and Russell, Molecular Cloning: A Laboratory Manual, 3^{rd} Ed. (Cold Spring Harbor Laboratory Press, Cold Spring Harbor, N.Y., 2001) and Current Protocols in Molecular Biology (Ausubel et al., eds., John Wiley & Sons, 1994-1998, Current Protocols, 1987-1994, as supplemented through July 2005 (Supplement 71)).

Transfections. For transfections, the cells were plated at a subconfluent density (5×10⁵/well) in wells of a 12 well plate. Eighteen hours later, cells were transfected with FuGene 6 reagent (Roche Diagnostics, Mannheim, Germany) that was mixed with plasmid DNA at a ratio of 1.5 μl/0.5 μg of total plasmid DNA for each transfection, according to the manufacturer's protocol. The day after transfection, the medium was replaced with one ml of fresh growth medium.

Isolation of cellular RNA. Total cellular RNA was isolated by using TRIZOL reagent (Invitrogen, Carlsbad, Calif., USA) according to the manufacturer's protocol and re-dissolved in water. Routinely, 0.5 ml of the TRIZOL reagent was used to extract RNA from the confluent monolayer of cells in a well of a 12-well plate.

RT-PCR. Samples of total RNA were treated with DNAse I (Ambion, Austin, Tex. USA) according to manufacturer's instructions. Residual DNAse was heat inactivated at 70° C. for 15 min. The DNAse-treated RNA was reversely transcribed by using oligo-dT polynucleotides and Mo-MLV reverse transcriptase (Invitrogen, Carlsbad, Calif., USA) according to the manufacturer's instructions. One tenth of the reversely transcribed RNA was amplified in a PCR reaction, by using TAQ DNA polymerase (Invitrogen Carlsbad, Calif., USA ) and the following reporter sequence-specific primers: (forward primer: 1: 5′-AAATACGAGATCCACCGAGACTCC-3′ (SEQ ID NO: 1) and reverse primer 2: 5′-GCAGGAACAGCGCCGATACAAT-3′ (SEQ ID NO: 2)). PCR conditions used were similar, or identical, to those described in U.S. patent publication No. 2006/0160108, which is incorporated herein by reference in its entirety. PCR reactions were performed on a ABI 9700 GENEAMP thermo-cycler.

Labeling of PCR products. One tenth of each completed PCR reaction was diluted with a fresh PCR reaction mixture containing 6-Carboxyfluorescein (6-FAM) 5′-labeled reporter polynucleotide-specific primer (primer 2: 5′-GCAGGAACAGCGCCGATACAAT-3′) and then incubated at 95° C. for 2 min, at 68° C. for 20 sec and at 72° C. for 10 min.

Endonuclease restrictions. Hpa I restriction endonuclease (New England Biolabs, Ipswich, Mass., USA) was directly added to the labeled PCR products at concentration of 5 U/reaction. The samples were digested for 2 hrs and purified using Qiaquick PCR purification columns (Qiagen, Hilden, Germany) according to the manufacturer's protocol.

Capillary electrophoresis. Serial dilutions of each Hpa I digested sample were analyzed by capillary electrophoresis using ABI PRIZM 3100 genetic analyzer (Applied Biosystems, Foster City, Calif., USA). A set of X-rhodamine-labeled MAPMARKER1000 molecular weight standards (Murfreesboro, Tenn. USA) was run in parallel to the analyzed samples as a molecular weight reference.

### 5.2. Example 2

The working examples below demonstrate the successful assessment of biological activities of sera derived from healthy and diabetic animals.

Experimental type I diabetes mellitus was induced in rats by using Streptozotocin (STZ), a N-nitroso derivative of D-glucosamine. Streptozotocin causes rapid necrosis in pancreatic β-cells of islets of Langerhans (Okamoto, 1985, Bioessays 2:15-21). STZ has been widely used to provoke insulin-dependent diabetes conditions in various laboratory animals, and animals treated with STZ are recognized in the art as a model of diabetes mellitus (Like and Rossini, 1976, Science 193:415-417).

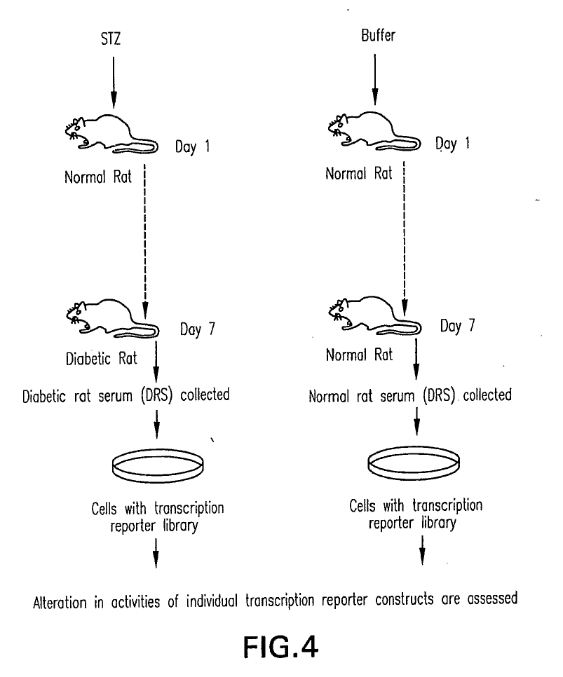

The design of the approach to compare sera from diabetic and healthy rats is illustrated in FIG. 4. Sprague-Dawley male rats aged 10-12 weeks were randomly allocated in two groups. Rats from one group received a single intra-peritonial injection of 70 mg/kg STZ (Sigma-Aldrich, St. Louis, Mo., USA) dissolved in freshly prepared 50 mM citrate buffer (pH, 4.0). Animals in control group received an equivalent volume of the citrate buffer. Seven days following the injection, blood samples were derived from a tail vein of the control and STZ treated animals. Development of the diabetic conditions in STZ treated animals was confirmed by blood glucose concentration measurements. Hyperglycemia (glucose levels of at least 300 mg/dl) was observed in all STZ treated rats, while glucose levels in all animals from the control group were normal (not more than 100 mg/dl).

Samples of normal rat serum (NRS) and diabetic rat serum (DRS) were obtained from five individual animals selected from control group and from five individual animals selected from STZ-treated group (day 7 after injection with STZ) using serum-separating tubes with clotting activator (Becton Dickinson, N.J., USA) according to the manufacturer's protocol.

A library of individual transcription reporter constructs, wherein each reporter contained a cis-regulatory element that was responsive to a particular TF, and each reporter RNA construct had a distinguishable reporter sequence, was constructed. The approach was used whereby each individual reporter construct is supplied with an identical reporter sequence that is supplied with a processing tag whose position varies within the library. The approach is described in detail in U.S. patent publication No. 2006/0160108, which is incorporated herein by reference in its entirety.

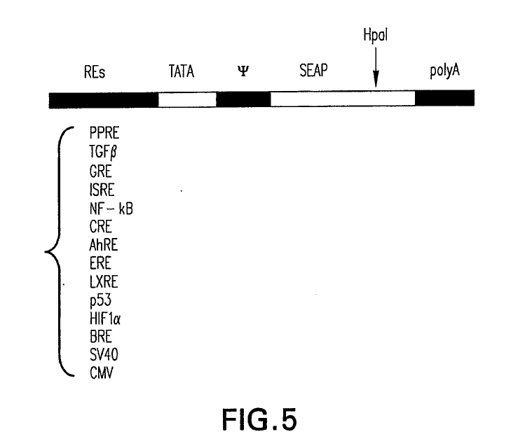

According to this approach, transcripts of individual reporters can be distinguished by processing (i.e., digesting) of the corresponding RT-PCR products at the position of the processing tag (a unique Hpa I digest site) followed by separation of the processed PCR products by electrophoresis. As shown by FIG. 5, the reporter library included the individual reporter RNA constructs with the following response elements (a.k.a. cis-regulatory sequences): peroxisome proliferator-activated receptor response element (PPRE), TGFβ-inducible response element (TGFβ), glucocorticoid receptor response element (GRE), interferon inducible response element (ISRE), NF-κB response element (NF-κB), cAMP response element (CRE), aryl hydrocarbon receptor response element (AhRE), estrogen receptor response element (ERE), liver X receptor response element (LXRE), p53 response element (p53), BMP-inducible response element (BRE), hypoxia-inducible factor 1 alpha response element (HIF-1α) an immediately early promoter from simian virus (SV40), and cytomegalovirus promoter (CMV). Each individual reporter RNA construct contained the reporter sequence that was identical, except that the HpaI cleavage site was introduced into individual reporter constructs at a unique variable position. Sequences of the response elements can be found, for example, in the following: PPRE (Evans et al., 2004, Nat. Med. 10(4):355-361); TGFβ (Dennler et al., 1998, EMBO J. 17(11):3091-3100); GRE (McEwan, 1997, Bioessays 19(2):153-160); ISRE (Kessler et al., 1990, Genes Dev. 4:1753-1765); NF-κB (Zabel et al., 1991, J. Biol. Chem. 266:252-260; Baldwin et al., 1991, Mol Cell Biol. 11:4943-4951); CRE (Benbrook and Jones, 1994, Nucleic Acids Res. 22:1463-1469); AhRE (McLane and Whitlock, 1994, Receptor 4(4):209-222); ERE (Naar et al., 1991, Cell 65(7):1267-1279); LXRE (Willy and Mangelsdorf, 1997, Genes & Development 11:289-298); p53 (Funk et al., 1992, Mol. Cell. Biol. 12(6):2866-71); BRE (Korchynskyi and Dijke, 2002, J. Biol. Chem. 277(7):4883-91); and HIF-1α (Miyazaki et al., 2002, J. Biol. Chem. 277:47014-47021). The complete details on the construction of the reporter constructs can be found in U.S. patent publication No. 2006/0160108, incorporated herein by reference in its entirety.

To create a transcription profiling system, the library of transcription reporter constructs was introduced into human hepatocellular carcinoma cell line, HepG2. To do so, HepG2 cells were plated into 12 well tissue culture plates at a density of 5×10⁶ cells/well. Next day after plating, the cells were transiently transfected with equimolar mix of the individual transcription reporter constructs. The transfected cells were maintained using normal growth medium (1 ml of DMEM with 10% fetal bovine serum/well) for two days.

To assess the biological activities induced by diabetic rat sera, normal growth medium was removed from the transfected cells and replaced with media (DMEM) supplemented with 20 percent of either normal rat serum or diabetic rat serum. Total RNA was extracted from the cells at two time points: 6 hours and 24 hours following the media change. The profiles of reporter trascripts were assessed as described in U.S. patent publication No. 2006/0160108. Briefly, the total RNA was reversely transcribed and amplified with a common pair of reporter sequence-specific primers, fluorescently labeled, processed (by digestion with the Hpa I restriction endonuclease), and resolved by using capillary electrophoresis. The relative activities of individual transcription reporter units were calculated as the values of corresponding individual peaks on the elctrophoregram and normalized on the mean value of all reporter peaks. Average values of each individual reporter construct obtained with five independent diabetic rat sera were compared with those obtained with five independent normal rat sera.

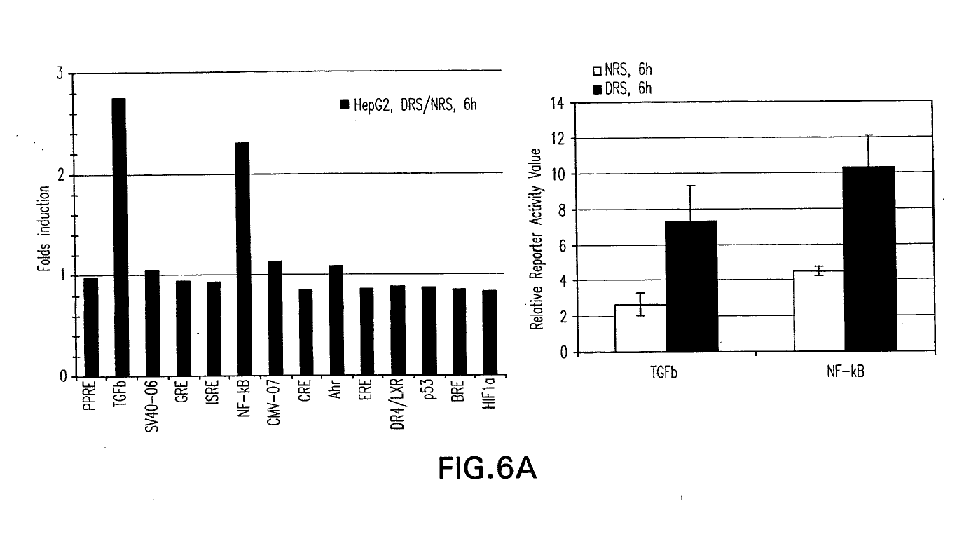

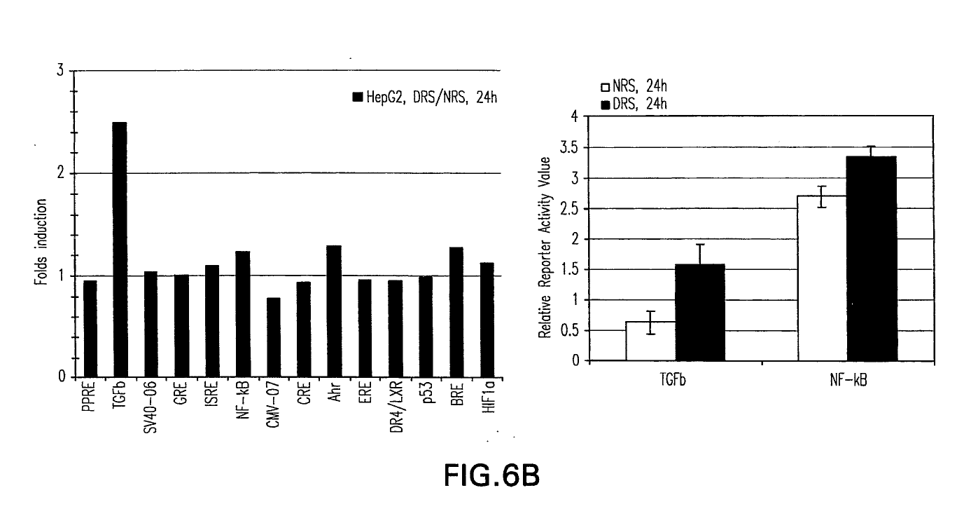

FIG. 6 shows the profiles of induction of activities of individual reporters in cells treated with diabetic rat sera normalized to the activities of the reporters in cells treated with normal rat sera. It was found that activities of two transcription reporter units, NF-kB and TGFβ, were significantly altered in cells exposed to diabetic sera. Increased levels of the TGFβ reporter transcription in cells treated with diabetic sera were evident at both experimental time points: 2.8-fold increase at six hours (FIG. 6A) and 2.7-fold increase at 24 hours (FIG. 6B) after addition of the sera. In contrast, alterations of the NF-kB transcription were transient. Thus, a 2.3-fold increase of the NF-kB reporter activity was evident only after 6 hours of incubation with diabetic sera, while at second time point, 24 hrs, relative activity of the NF-kB reporter construct was not significantly different in cells treated with diabetic sera and in control samples. Notably, the profiles of transcriptional activities induced by the individual sera within each group (normal or diabetic) were very consistent, with standard deviation of each reporter construct activity not exceeding 20% of the average value of the reporter construct activity measured across each group. Thus, it was concluded that sera derived from STZ-induced diabetic rats can be distinguished from the sera of normal animals on the basis of their effects on activities of transcription reporter constructs in HepG2 cells.

### 5.3. Example 3

This example demonstrates that detected transcriptional activities can be specific to the type of cell utilized as a biosensor cell.

Human kidney epithelial cells, HEK293, and rat β-cellular insulinoma cell line, U7, were transiently transfected with the library of transcription reporter construct as described for HepG2 cells in Example 2. Two days later, the HEK293 and U7 biosensor cells were exposed to the media containing either diabetic or healthy rat serum for six hours. The procedures followed were similarly those as described in Example 2 above and in U.S. patent publication No. 2006/0160108, which is incorporated herein by reference in its entirety. Briefly, the total RNA was reversely transcribed and amplified with a common pair of reporter sequence-specific primers, fluorescently labeled, processed (by digestion with the Hpa I restriction endonuclease), and resolved by using capillary electrophoresis. The relative activities of individual transcription reporter constructs were calculated as the values of corresponding individual peaks on the elctrophoregram and normalized on the mean value of all reporter peaks. Average values of each individual reporter construct obtained with five independent diabetic rat sera were compared with those obtained with five independent healthy rat sera.

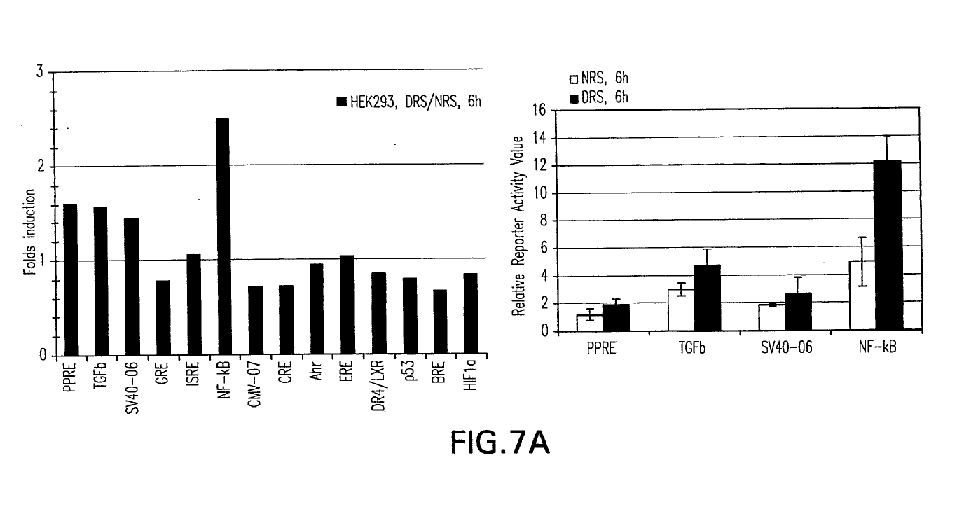

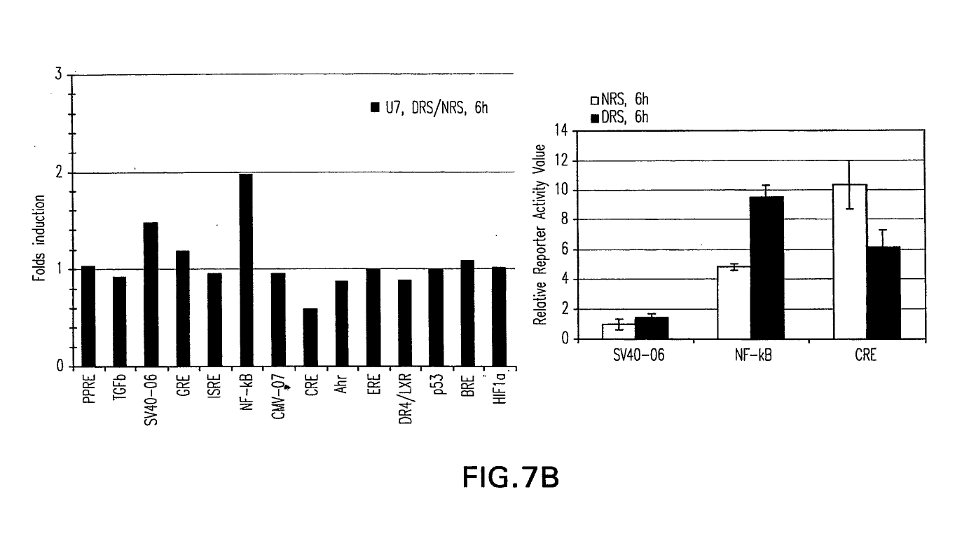

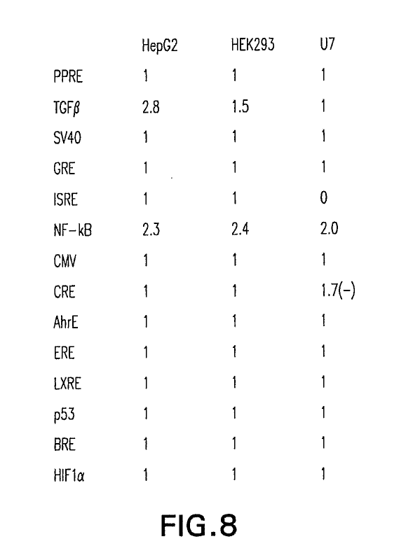

FIGS. 7A and 7B show the profiles of induction/down-regulation of activities of individual reporters in, respectively, HEK293 and U7 biosensor cells treated with diabetic rat sera normalized to the activities of the reporters in reference cells treated with healthy rat sera. The profiles of transcriptional responses induced by diabetic sera at 6 hours in three different cell lines were diverse (compare FIGS. 7A, FIG. 8A and FIG. 8B). NF-kB was the only transcription reporter construct, activity of which was consistently induced by diabetic serum in all biosensor cell lines. The degree of the NF-kB induction was also similar across all types of the biosensor cells: 2.3-fold induction in HepG2 cells, 2.4- fold induction in HEK293 cells and 2.0-fold induction in U7 cells. Increase of TGFP reporter activity was limited to HepG2 (2.8-fold) and HEK293 (1.5-fold) biosensor cells. SV40 transcription was induced by diabetic serum in HEK293 (1.6-fold) and in U7 (1.8-fold) cells, but not in HepG2 cells. Noticeable down-regulation of CRE transcription was only observed in U7 cells (1.7-fold decrease). Thus, alterations of transcription reporter profiles induced by sera of diabetic animals are cell-type specific.

### 5.4. Example 4

This example demonstrates the assembly of data into a matrix form useful, for example, as a standard of comparison for diagnosing experimental diabetes in rats.

In examples 2 and 3, it was demonstrated that sera obtained from diabetic and normal animals can be distinguished on the basis of the differential alterations they exert in the profile of activities of transcription reporter constructs.

Transcriptional responses that are differentially induced by the sera extracted from diabetic animals in a variety of reporter cell types can be organized in a form of matrix, wherein each column represents profile of induction of library of transcription reporter constructs in individual reporter cell type, and, respectively, each raw represents profile of induction of individual transcription reporter construct across different reporter cell types. In this matrix, each pair of (reporter cell: reporter construct) is assigned a value that is equal to of fold-induction of average activity of given reporter construct in given reporter cell type by diabetic rat sera normalized to the average activity of given reporter construct in given reporter cell type treated by normal rat sera. If the average activity of given reporter construct in given reporter cell type treated with diabetic sera is not significantly different from the activity of given reporter construct in given reporter cell type treated with normal rat sera, the assigned value is equal 1. The significance of change in the activity of given reporter construct induced by diabetic sera may be assessed by using any standard statistical algorithm. Any one knowledgeable in the art will understand that result will also depend on number of individual diabetic and normal sera used and on variability of the responses exerted by different sera. For the purpose of the current example, the following criteria of significance of alteration of transcription reporter activity induced by diabetic sera were set: 1) the mean value of the activity in the presence of diabetic sera should be at least 1.5-fold different from that in the presence of normal sera; 2) spread of individual variations around the mean value (standard deviation) of activity measured in the presence of diabetic and normal sera should not overlap. FIG. 8 illustrates an example of the prototypic matrix of significant transcriptional responses induced by diabetic sera in HepG2, HEK293 and U7 reporter cell lines, assembled based on the data shown in FIG. 6 and FIG. 7 (6 hours time points).

The prototypic matrix shown in FIG. 8, can be easily extended by 1) expanding the library of individual transcription reporter units, and 2) by broadening the list of reporter cell types. Knowledgeable in the art will understand that composition and threshold of the significant alterations included in the matrix may change when size of the experiment (i.e. number of individual sera analyzed) is increased.

The matrix of transcription responses similar to that shown in FIG. 7 provides a unique molecular signature of the STZ-induced diabetic condition. It can be used for the disease identification purposes.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed:

1. A method of determining biological activities of a biological sample, comprising:
contacting said biological sample with biosensor for a defined time;
assessing the profile of activities of transcription factors (TFs) within said biosensor;
comparing the profile of activities of transcription factors (TFs) with the profile of activities of TFs in the biosensor that was not contacted with the biological sample; and
generating the differential profile of alterations of TF activities that represents the biological activity of the evaluated sample.

2. The method of claim 1, wherein the profile of TF activities represents activities of more than one TF.

3. The method of claim 1, wherein the profile of TF activities comprises activities of 2, 5, 10, 20, 40, 100, 200, 1,000, or 2,000 TFs.

4. A method of determining biological activities of a biological sample comprising
contacting said biological sample with biosensor;
assessing temporal profile of activities of a TF within said biosensor at different time points after the contact;
comparing the temporal profile of activities of the TF with the temporal profile of activities of the TF in the biosensor that was not contacted with the biological sample,
and generating the differential temporal profile of alterations of TF activities that represents biological activity of the evaluated sample.

5. The method of claim 4, wherein the profile of TF activities represents activities of more than one TF.

6. The method of claim 4, wherein the profile of TF activities comprises activities of 2, 5, 10, 20, 40, 100, 200, 1,000, or 2,000 TFs.

7. The method of claim 4, wherein the profile comprises TF activities determined at more than one time point.

8. The method of claim 4, wherein the profile comprises TF activities determined at 2, 5, 10, 20, 40, 100, or 200 time points.

9. A method of determining biological activities of a biological sample comprising
contacting said biological sample with a panel of biosensors;
assessing the alterations of TF activities within individual biosensors in response to the contact;
and generating the profile of alterations of TF activities within the plurality of biosensors, thereby characterizing the biological activity of the evaluated sample.

10. The method of claim 9, wherein the panel of biosensors represents more than one biosensor.

11. The method of claim 9, wherein the panel of biosensors comprises 2, 5, 10, 20, 40, 100, or 200 individual biosensors.

12. The method of claim 9, wherein the individual biosensors comprise different cell types, including fibroblasts, epithelial cells, immune cells, neural cells, stem cells, etc.

13. The method of claim 9, wherein individual biosensors comprise different tissue cultures, organ cultures, cell and tissue cultures engrafted into animals, organs and tissues of a live animal, and whole live animals.

14. The method of claim 1, 4 or 9, wherein the profile of activities of TFs are the DNA-binding activities of said TFs.

15. The method of claim 1, 4 or 9, wherein the biosensor is supplied with reporter gene constructs enabling assessing the transcriptional activities of said TFs and cis-regulatory elements (cisREs) regulated by TFs.

16. The method of claim 15, wherein the cisRE is a DNA sequence of a gene promoter, a gene enhancer, an RNA stability determinant, a naturally occurring or a synthetic DNA sequence whose transcriptional activity is modulated by contact with evaluated sample.

17. The method of claim 15, wherein the reporter gene construct is designed to assess the transcriptional activity of a chimeric TF representing a fusion of transactivating domain of one TF with DNA-binding domain of another protein.

18. The method of claim 1, 4 or 9, wherein the biological biosensor is a plurality of cell cultures, a mixed population of cells, a tissue culture, an organ culture, an engrafted cell and tissue culture, an organ or a tissue of a live animal.

19. The method of claim 1, 4 or 9, wherein said sample comprises biological fluid (saliva, blood, serum, cerebrospinal fluid, synovial fluid, urine, semen, breast milk, bile, tears, feces, extracts, etc.), as well as extracts, concentrates, components, or fractionates thereof.

20. The method of claim 1, 4 or 9, wherein said sample comprises cell and tissue extracts, conditioned cell culture media, etc.

21. The method of claim 1, 4 or 9, wherein said sample comprises live or fixed cells.

22. The method of claim 1, 4 or 9, wherein when biosensor is contacted with a combination of the sample and a response-modifying agent.

23. The method of claim 22, wherein where the modifying agent is a cytokine, a low-molecular weight compound, a small interfering RNA, a gene expression vector, an antisense oligonucleotides, a radiation, or any other treatment that can alter the TF activity profile of biosensor.

24. A method of describing functional state of a biological system by determining molecular signatures of biological activities of a single or multiple samples derived from said biological system as described in claims 1, 4, and 9.

25. A method of identifying markers of perturbed functional state of a biological system by:
determining biological activities of a single or multiple biological samples derived from said perturbed organism as described in claims 1, 4, and 9,
determining biological activities derived from unperturbed organism as described in claims 1, 4, and 9, and
comparing said biological activities and generating the differential molecular signature characterizing the perturbation.

26. A method of evaluating the intensity of perturbation by quantitatively evaluating the intensity of the perturbation-associated biological activities in evaluated samples.

27. The method of claim 25, wherein said perturbation is a disease, pre-disease state, aging, physical treatment, stress, diet, therapeutic treatment, administration of chemical compounds, toxins, pathogens, etc.

28. The method of claim 25, where biological system is cell culture, a mixed population of cells, a tissue culture, an organ culture, an engrafted cell and tissue culture, an organ or a tissue of a live animal, or a whole live animal.

29. The method of claim 25, where biological system is human.

30. A method of identifying differential markers distinguishing two perturbed functional states of a biological system by
determining biological activities of a single or multiple biological samples derived from from one perturbed state of organism as described in claims 1, 4, and 9,
determining biological activities of a single or multiple biological samples derived from from another perturbed state of organism as described in claims 1, 4, and 9, and
comparing said biological activities and generating the differential molecular signature that distinguishes the perturbed states.

31. A method of diagnostics of disease and pre-diseased states of organism by determining biological activities of a single or multiple biological samples derived from evaluated organism as described in claims 1, 4, and 9, and matching the biological activities of said samples with database of markers of perturbed functional states as described in claim 25.

32. The method of claim 31, where the disease is an inflammatory disease, a metabolic disease, cancer, a neurodegenerative disease, a psychosomatic disease, an infection, a premature aging, etc.

33. A method of identifying therapeutic modalities for a disease or a pre-disease state by
identifying markers of a disease or a pre-disease state as in claim 25,
identifying the TFs whose activities are differentially regulated in the disease or in the pre-disease state,
altering the activities of those TFs toward their activities in the unperturbed state.

34. A method of selecting drug candidates with desirable therapeutic properties by
treating perturbed biological system with evaluated drug candidate
assessing the alterations of biological activities in biological samples derived from the perturbed system in response to the treatment and
selecting the drug candidates that normalize the biological activities of samples toward that in unperturbed biological system.

35. The method of claim 34, where biological system is cell culture, a mixed population of cells, a tissue culture, an organ culture, an engrafted cell and tissue culture, an organ or a tissue of a live animal, or a whole live animal.

36. A method of identifying markers of individual variations of responses to therapeutic treatments by
administering said therapeutic treatment to a group of patients;
dividing the treated group into one group, that produces desirable outcome of said treatment, and another group, that produces undesirable outcome of said treatment;
collecting biological samples prior to and after said treatment from the group of patients that shows desirable therapeutic outcome of said treatment, and determining markers of common alterations in the biological activities of the specimens in response to said treatment;
collecting biological samples prior to and after said treatment from the group of patients that shows undesirable therapeutic outcome of said treatment, and determining markers of common alterations in the biological activities of the specimens in response to said treatment; and
identifying the differential markers associated with desirable outcome of the treatment.

37. A method of predicting individual response to a therapeutic treatment by
subjecting patient to the therapeutic treatment;
collecting biological samples prior to and after said treatment and determining alterations in the biological activities of the samples in response to the treatment, and
comparing treatment-inducible alterations in the biological activities of the samples with database of biological activities of patients that show desirable and undesirable therapeutic outcome of said treatment as in claim 36.

38. The method claim 36 or 37, where therapeutic treatment comprises any treatment directed toward altering the physiological state of organism, including, but not limited to, natural and synthetic drugs, radiation, surgery, gene therapy, hypnosis, manual therapy, etc.
